(19) United States
(12) Patent Application Publication
(10) Pub. No.: US 2015/0159180 A1
(43) Pub. Date: Jun. 11, 2015

(54) PROCESS FOR PRODUCTION OF CRYSTALLINE XYLITOL USING PICHIA CARIBBICA AND ITS APPLICATION FOR QUORUM SENSING INHIBITION

(71) Applicant: COUNCIL OF SCIENTIFIC & INDUSTRIAL RESEARCH, New Delhi (IN)

(72) Inventors: Asmita Ashutosh Prabhune, Maharashtra (IN); Kasturi Laxman, Maharashtra (IN); Ruchira Arup Mukherji, Maharashtra (IN)

(73) Assignee: COUNCIL OF SCIENTIFIC & INDUSTRIAL RESEARCH, New Delhi (IN)

(21) Appl. No.: 14/414,834

(22) PCT Filed: Jul. 16, 2013
(86) PCT No.: PCT/IN2013/000438

§ 371 Date: Jan. 14, 2015

(30) Foreign Application Priority Data
Jul. 16, 2012 (IN) 2190/DEL/2012

(51) Int. Cl. C12P 7/18; C07C 27/26

(52) U.S. Cl. CPC C12P 7/18; C07C 27/26

(57) ABSTRACT

The inventors disclose a novel room temperature process for the synthesis of crystalline xylitol with high yield and purity from D-xylose using Pichia caribbica yeasts that acts as a quorum sensing antagonist that prevents bio film formation by gram-negative bacteria. Further a mild and safe procedure for xylitol extraction is disclosed.

### FIELD OF THE INVENTION

The present invention relates to a fermentation process for production of xylitol from D-xylose using a novel yeast species, Pichia caribbica BY2 (deposited at MTCC, IMTECH, Chandigarh and having accession no. MTCC 5703), involving an improved downstream processing for separation of xylitol from its fermentation broth in pure crystalline form and in quantitative yield. Particularly, present invention relates to the use of Pichia caribbica BY2 synthesized xylitol as a quorum sensing antagonist in gram negative organisms.

### BACKGROUND OF THE INVENTION

Xylitol is a naturally occurring five carbon sugar alcohol (pentanol) belonging to the polyol group commonly found in fruits and some vegetables. With the expanding market of artificial sweeteners xylitol is fast gaining ground due to its many advantages over other artificial sugar substitutes. Xylitol is as sweet as sugar (sucrose) but due to its insulin independent metabolism and low glycemic index it has been widely used as an ideal sweetener for diabetic patients.

A variety of interesting applications have been reported for Xylitol especially in the field of odontology. A notable anti-biofilm effect of Xylitol has been recognized against Streptococcus mutans, the organism associated with dental decay. The polysaccharide capsule of S. mutans plays major role in tooth enamel adherence and subsequent formation of biofilm in the form of dental plaque. The capsule synthesis occurs through the utilization of dietary sugars like sucrose. D-Xylitol cannot be utilized by S. mutans and thus capsule synthesis and further consequences are avoided. Due to the anti-biofilm activity and negative heat of solution associated with xylitol, it has been shown to prevent tooth decay, cause tooth rehardening and remineralization and hence forms an important component of toothpastes, mouthwashes and sugar free chewing gums.

Xylitol is also known to possess antimicrobial activity. It averts ear and upper respiratory infections caused by Streptococcus pneumoniae, which is responsible for 30% or more of such attacks. It also suppresses Haemophilus influenzae, another important pathogen implicated in these kinds of infections.

Most published prior arts disclose the antipathogenic effect of xylitol against gram positive organisms. Effect of xylitol on gram negative organism has still not been established.

The global market demand for polyols is expecting annual 2.7% increase to reach 1597000 metric tons. The advantageous physical and chemical properties of xylitol make it a highly sought after compound for pharmaceutical, odontological and food industries.

In nature Xylitol is abundantly found in fruits and vegetables such as berries, corn husks, oats, lettuces, cauliflowers, and mushrooms but its extraction from them is difficult owing to the fact that the resultant yield is very low.

D-Xylitol is industrially produced by chemical hydrogenation of pure synthetic xylose at high temperature and under high pressure of about 50 atm using raney nickel as catalyst.

EP2314560 relates to a method of producing xylitol comprising hydrolyzing a D-glucuronic acid compound, decarboxylating a salt of the D-glucuronic acid compound to produce a dialdehydo xylitol intermediate, and hydrogenating the dialdehydo xylitol intermediate in the presence of a hydrogenation catalyst to produce xylitol.

U.S. Pat. No. 5,714,602 relates to a method of producing xylitol from gluconic acid which comprises decarboxylating the gluconic acid to give an intermediate consisting mainly of arabinose, hydrogenating the arabinose in the presence of a catalyst to give the corresponding pentitol, arabinitol, catalytically isomerizing the arabinitol to a xylitol-containing pentitol mixture, separating xylitol from the pentitol mixture to obtain a residual pentitol mixture, and optionally recycling of the residual pentitol mixture.

JP62277332 relates to a process for production of xylitol wherein a xylan-containing natural substance is steamed or boiled or ground by explosion at 130-230° C. for 1-60 min, extracted with water or warm water to give an aqueous solution, which is hydrolyzed and simultaneously hydrogenated in the presence of Raney nickel catalyst or ruthenium catalyst at 150-200° C. for 60-180 min at 50-150 kg/cm² hydrogen pressure. The catalyst is removed from the reaction solution and purified by a conventional procedure such as deionization, etc., concentrated and crystallized to give xylitol crystal.

The production of xylitol in the above existing state of art involves chemical processes which are harsh, laborious, costly and energy intensive: The processes also require expensive refining treatment in order to produce pure xylitol. Furthermore, yield of product obtained from the chemical hydrogenation is about 50-60%. Hence alternative methods involving fermentation by microorganisms and also enzymatic conversion of D-Xylose to Xylitol operating at ambient conditions are currently being explored.

Microbial sources have been exploited extensively for xylitol production; among them yeasts have been touted to be the best Xylitol producers from hemicellulosic or other xylan rich biomass among all the microorganisms investigated. Yeasts are especially favored because they are very robust organisms and can easily grow on inexpensive renewable resources like hemicellulose. Yeast of genera Candida, Debaryomyces and Pichia have been reported to give good Xylitol yield from their fermentation medium.

US20040191881 discloses a submerged fermentation process for production of xylitol from Pichia sp. using cotton seed flour as cheap organic nitrogen source. However, said US publication does not disclose downstream process for the purification of xylitol.

U.S. Pat. No. 5,081,026 discloses a method for the production of xylitol from an aqueous solution containing xylose. The aqueous xylose solution is fermented with a yeast strain, Debaryomyces hansenii which is capable of converting the free xylose present to xylitol. The xylitol-rich fraction is separated by chromatographic separation from said fermented solution; and xylitol is recovered from said fraction.

U.S. Pat. No. 5,686,277 discloses a fermentation process for preparing xylitol with high productivity using novel mutant cells of Candida parapsilosis by optimizing the composition of medium containing xylose and the environmental conditions of culture such as pH, temperature and DO concentration, anal controlling the concentration of mutant cells.

An article titled “Downstream processing for xylitol recovery from fermented sugar cane bagasse hydrolysate using aluminium polychloride” by silva et.al. published in Z Naturforsch. C. 2000 January-February; 55(1-2):10-5 discloses xylitol recovery from fermented sugar cane bagasse hydrolysate using 5.2 g/l of aluminium polychloride associated with activated charcoal. Aluminium polychloride and activated charcoal promoted a 93.5% reduction in phenolic compounds and a 9.7% loss of xylitol from the fermented medium, which became more discolored, facilitating the xylitol separation.

The fermentation processes as disclosed in the art however produces relatively less pure xylitol. Moreover, crystallization of xylitol is difficult and the downstream processing for the recovery of xylitol used in the art is costly and cumbersome. Furthermore, prior art processes require charcoal treatment or ion-exchange chromatography for downstream processing and for decolorization of xylitol. This makes the process less cost effective and also results in low yield.

### OBJECT OF THE INVENTION

Main object of the present invention is to provide a process for quantitative production of xylitol from pure D-xylose using a novel yeast species, Pichia caribbica BY2 (NCBI Genbank database accession no. HQ222812, MTCC 5703).

Another object of the present invention is to provide a mild method of extraction of synthesized xylitol from the fermentation broth with 99% purity in crystalline form and with almost quantitative yield.

Yet another object of the present invention is to use Pichia synthesized xylitol as a quorum sensing antagonist in gram negative organisms.

### SUMMARY OF THE INVENTION

Accordingly, the present invention provides a room temperature fermentation process for the synthesis of crystalline xylitol from D-xylose using yeast strain Pichia Caribbica BY2 having MTCC No. 5703 comprising the steps of:
i. inoculating 10⁸ cells per ml of MXYP (malt extract xylose yeast extract and peptone medium) with yeast strain Pichia caribbica BY2 and incubating at temperature in the range of 25 to 28° C. in a rotary shaker at 170 to 180 rpm for period in the range of 20 to 24 hours to obtain inoculated MXYP broth;
ii. adding the inoculated MXYP broth as obtained in step (a) into 90 ml MXYP broth and further incubating for period in the range of 40 to 48 hours to get sufficient cell biomass;
iii. harvesting cells from MYXP broth by centrifugation at 4500 to 5000 rpm for period in the range of 15 to 20 minutes at temperature in the range of 4 to 10° C.;
iv. re-dispersing the cells from step (c) in the ratio ranging between 3 to 4 g % of 9.5 to 10% D-Xylose solution and incubating further for period in the range of 70 to 72 hr;
v. separating cells by centrifugation at 4500 to 5000 rpm for period in the range of 15 to 20 mins at temperature in the range of 4° C. to 10° C.;
vi. concentrating the yellow coloured supernatant obtained in step (e) by rotary vacuum evaporation in a 85° C. water bath to obtain the bright yellow viscous fluid;
vii. further concentrating the bright yellow viscous fluid obtained as obtained in step (f) by centrivac concentrator system;
viii. crystallizing the concentrated bright yellow viscous fluid as obtained in step (g) by refrigerating at temperature in the range of (−)15° C. to (−)20° C. for period in the range of 40 to 48 hours, to obtain semi crystalline yellow material;
ix. decolourizing the semi-crystalline yellow coloured material as obtained in step (h) using absolute ethanol to obtain crystalline xylitol with 0.8 to 0.9 gm/gm yield and 98 to 99% purity.

In one embodiment of the present invention, the MXYP broth used for inoculating Pichia caribbica BY2 contains Malt extract 0.3 g % to 0.33 g %, Yeast extract—0.3 g % to 0.33 g %, Mycological peptone,—0.6 g % to 0.65 g % and D-Xylose—2 g % to 2.5 g %.

In an embodiment of the present invention, said crystalline Xylitol acts as a quorum sensing antagonist that prevents the bio film formation by gram negative bacteria using anti-AHL (Anti-acyl homoserine lactone) activity of xylitol.

In another embodiment of the present invention, the gram negative system is preferably Chromobacterium violaceum CV026.

In yet another embodiment of the present invention, said xylitol inhibits Quorum sensing signal (acyl homoserine lactone) reception by three distinct signal molecules selected from C6-HSL, C8-HSL and 3-oxo-C6-HSL in gram negative bacteria.

In yet another embodiment of the present invention, said xylitol acts as a quorum sensing antagonist in Burkholderia cenocepacia having two distinct signal molecules, C6-HSL and C8-HSL or in Erwinia carotovora having one signal molecule, (3-oxo-C6-HSL).

In yet another embodiment of the present invention, room temperature is in the range of 25 to 28° C.

### DETAILED DESCRIPTION OF THE DRAWINGS

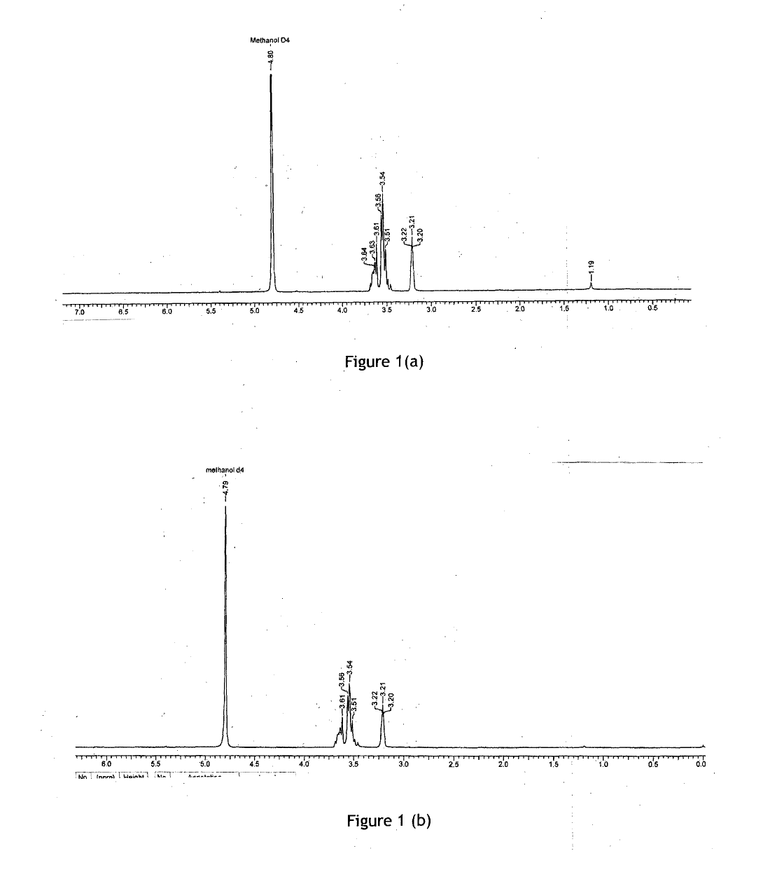

FIG. 1a: H¹ NMR spectra of Sigma Xylitol.

FIG. 1b: H¹ NMR spectra of Pichia synthesized Xylitol.

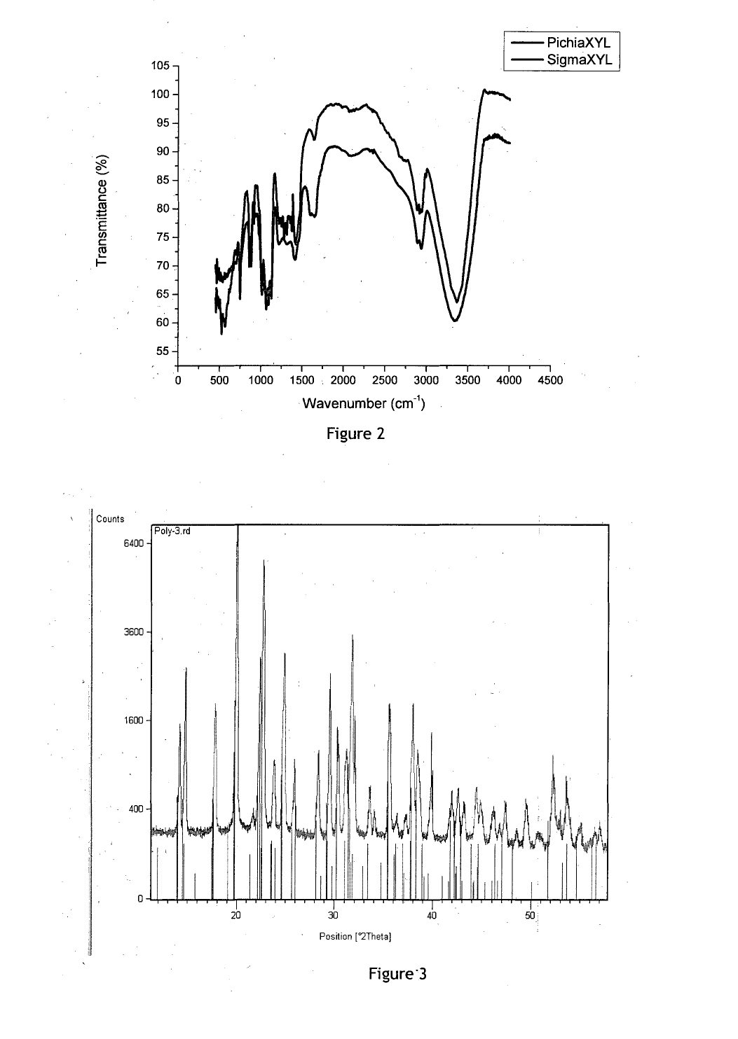

FIG. 2: Fourier transform-IR spectra of Pichia synthesized xylitol and pure Sigma xylitol.

FIG. 3: Graph representating of the XRD data.

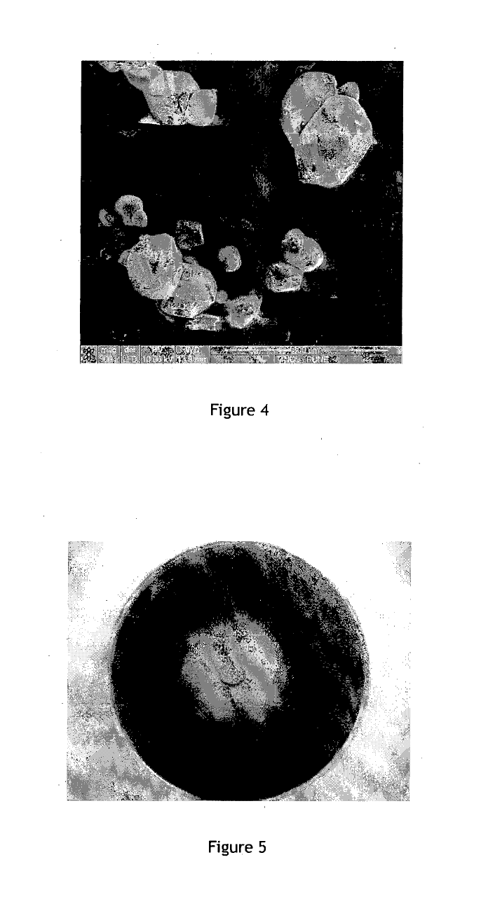

FIG. 4: Crystal morphology of Pichia synthesized xylitol as seen under the scanning electron microscope.

FIG. 5: Photograph of agar plate showing zone of inhibition of violacein synthesis as seen around the agar well containing Pichia synthesized xylitol.

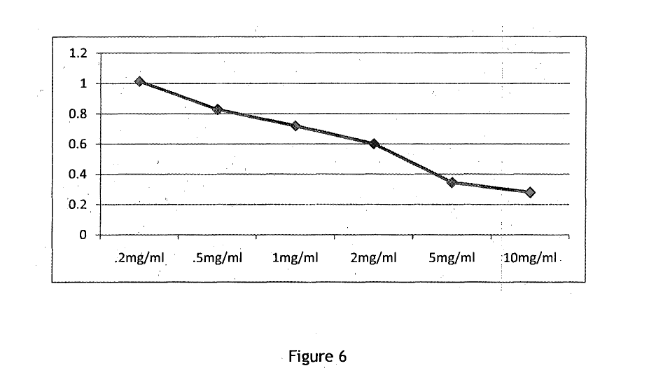

FIG. 6: Quantitative inhibition of violacein synthesis in the presence of increasing concentration of Pichia synthesized xylitol.

### DETAILED DESCRIPTION OF THE INVENTION

Present invention provides a fermentation process for production of xylitol from D-xylose using a novel yeast species, Pichia caribbica BY2 (MTCC 5703). The synthesized xylitol from the fermentation broth is separated and purified using a mild, cost effective downstream processing to obtain crystalline xylitol which is 99% pure and in almost quantitative yield.

The invention discloses the use of Pichia synthesized xylitol as a quorum sensing antagonist in gram negative organisms.

The novel yeast species, Pichia caribbica BY2 (MTCC 5703), of the present invention is isolated from over ripe banana by enrichment of xylose-containing medium. A piece of over-ripe banana is inoculated in an enrichment medium containing xylose, malt extract, yeast extract and mycological peptone and incubated for 48 hours at 28° C. on a rotary shaker at 180 rpm. All the yeast isolates are screened for xylose fermentation. The xylose fermenting yeast, designated as. BY2, is selected and further purified by picking up a single colony. DNA is isolated from isolate BY2 using the colony lysis method and PCR amplified using primers specific for the D1/D2 and ITS regions. The contig obtained is deposited in the NCBI Genbank database under the accession no. HQ222812. This is followed by phylogenetic analysis. The phylogenetic analysis of ITS1-5.8S-ITS2 region sequences of ribosomal RNA of isolate BY2 reveals that it shows affiliation to genus Pichia and clades with Pichia caribbica.

The present invention relates to a fermentation process for the production of crystalline xylitol from pure D-xylose using a novel yeast species, Pichia caribbica BY2 in good yield and with 99% purity.

The Pichia caribbica BY2 synthesized xylitol is separated and purified from the fermentation broth using a mild, cost effective downstream process to obtain crystalline xylitol which is 99% pure and in almost quantitative yield.

The downstream process involves simple rota-vaporization and concentration of the spent broth which contains high amount of synthesized xylitol (almost quantitative production), followed by a low temperature storage for 48 hours to allow, crystal growth. The product crystals in this case are associated with yellow pigment which is completely removed by treatment with 100% ethanol.

Accordingly, the fermentation process for production of 99% pure crystalline xylitol from D-xylose using Pichia caribbica BY2, involving downstream process, comprises;
(a) inoculating 10 ml of MXYP with yeast strain Pichia caribbica BY2 and incubating at 28° C. in a rotary shaker at 180 rpm for 24 hours;
(b) adding the inoculated MXYP broth of step (a) into 90 ml MXYP broth and further incubating for 48 hours to get sufficient cell biomass;
(c) harvesting cells from MXYP broth by centrifugation at 5000 rpm for 20 minutes at 10° C.;
(d) re-dispersing the cells from step (c) in 100 ml of 10% D-Xylose solution and incubating further till 72 hr;
(e) separating cells by centrifugation at 5000 rpm for 20 mins at 10° C.;
(f) concentrating the yellow coloured supernatant obtained in step (e) by rotary vacuum evaporation in a 85° C. water bath;
(g) further concentrating the bright yellow viscous fluid obtained in step (f) by centrivac concentrator system;
(h) crystallizing the bright yellow viscous fluid of step (g) by refrigerating at −20° C. for 48 hours, and
(i) Decolourizing the semi-crystalline yellow coloured material using absolute ethanol to obtain xylitol in complete dry white crystalline powder form.

The preferred pentose sugar containing source is D-xylose. The xylose or other pentose sugar containing raw materials such as hemicellulose also stand as potential source for xylitol production. However depending upon the composition of raw material, additional downstream processing steps may be required to achieve crystalline product

The MXYP broth used for inoculating Pichia caribbica BY2 contains the following ingredients:
Malt extract—0.3 g %
Yeast extract—0.3 g %
Mycological peptone—0.6 g %
D-Xylose—2 g %

Accordingly, 10 ml of MXYP broth is inoculated with Pichia caribbica BY2 cells. This is incubated at 28° C. in a rotary shaker 180 rpm for 24 hours. After incubation the MXYP broth is added to 90 ml MXYP broth and further incubated for 48 hours at 28° C. in a rotary shaker at 180 rpm.

After 48 hours, the cells are harvested from MXYP broth by centrifugation at 5000 rpm for 20 minutes at 10° C. The harvested cells, having a wet weight of approximately 3.6 g, are then re-dispersed in 100 ml of 10% xylose solution, which serves as the production medium for xylitol and incubated further till 72 hours.

After 72 hours the cells are separated by centrifugation at 5000 rpm for 20 mins at 10° C.

The bright yellow coloured supernatant is collected and concentrated by rotary vacuum evaporation in a water bath maintained at 85° C. The concentrate obtained in step (f) is further concentrated by centrivac system.

The condensate obtained in step (f) is subjected to HPLC analysis for residual xylose determination. And 90% xylose utilization was confirmed. After the fermentation is completed, any residual sugar is checked. The cells obtained after fermentation can be reused for up to 3 times without substantial loss of efficiency.

The decolourization in step (i) is done by adding 5 ml of absolute ethanol to 2 g of semi-crystalline yellow coloured material. The material is washed thrice with the absolute ethanol over a period of 24 hrs and once again after overnight contact. The intense colour gets eluted from the crystals into the solvent (ethanol) layer. The solvent is discarded and the residual solvent is allowed to evaporate at room temperature to get complete dry white crystalline powder.

The crystalline white compound obtained from the spent broth is determined to be xylitol using various characterization techniques. The purity of the Pichia synthesized xylitol is determined qualitatively using HPLC. Xylitol yield obtained is 85 g/l, which is close to the maximum reported till now using yeasts as microbial factories.

The Pichia synthesized crystalline xylitol of the current invention is further characterized by XRD, H¹NMR, FT-IR analysis, HPLC analysis, and Scanning Electron Microscopy (SEM).

In yet another embodiment, the present invention provides the use of the Pichia synthesized xylitol to act as a quorum sensing antagonist in gram negative organisms.

The Pichia synthesized xylitol is tested as a quorum sensing inhibitor in gram negative systems by anti-AHL (Anti-acyl homoserine lactone) activity of xylitol. Chromobacterium violaceum CV026 is used as the test organism for testing the anti-AHL activity since it serves as a representative gram negative system capable of responding to many natural and synthetic AHL molecules. The Pichia synthesized xylitol inhibits Quorum sensing signal (acyl homoserine lactone) reception by three distinct signal molecules namely C6-HSL, C8-HSL and 3-oxo-C6-HSL all of which serve as important signaling molecules in virulence gene regulation of various pathogenic gram negative bacteria.

Acyl homoserine lactones (AHL) are the largest class QS signals which play a crucial role in gram negative cell to cell signalling and virulence gene expression. Pichia synthesized xylitol is able to antagonize not one but three different AHL signal and receptor interaction as determined using CV026 based AHL antagonism assay system. All of these molecules C6-HSL, C8-HSL and 3-oxo-C6-HSL are important signaling components for virulence genes activation in major gram negative pathogens.

For example, Burkholderia cenocepacia, an important human pathogen which causes pulmonary infection in immune compromised patients and individuals with cystic fibrosis, uses both C6-HSL and C8-HSL, as its QS signal molecule to activate virulence and biofilm formation genes. Also plant pathogens like Erwinia carotovora synthesize exoenzyme like pectinases and exopolysaccharide in response to its quorum sensing signal molecule 3-oxo-C6-HSL which is one of the signal molecule antagonized by xylitol.

### EXAMPLES

Following examples are given by way of illustration and therefore should not be construed to limit the scope of the invention.

### Example 1

Isolation and identification of yeast species, Pichia caribbica BY2 (MTCC 5703) by method reported in inventor's own publication entitled “Evaluation of ethanol production by a new isolate of yeast during fermentation in synthetic medium and sugarcane bagasse hemicellulosic hydrolysate” by Aparna Hande et al.

### Isolation

A piece of over-ripe banana was inoculated in enrichment medium containing (g l⁻1): xylose 40, malt extract 3, yeast extract 3 and mycological peptone 5, pH 6.0±0.2, and incubated at 28° C. on a rotary shaker at 180 rpm. After enrichment for 48 h, organisms were isolated from enrichment culture broth by direct plate method on the agar medium. All the yeast isolates were screened for xylose fermentation. The xylose fermenting yeast was further purified by picking up a single colony, and pure culture was maintained on a medium similar to the enrichment medium but solidified using 20 g l⁻¹ agar.

### DNA Isolation, Amplification and Purification

DNA isolation was done using the colony lysis method. A colony of isolate BY2 was suspended in 20 μl Tris EDTA, incubated at 95° C. for 5 min, snap chilled in ice, and centrifuged at 10,000 rpm for 3 min. The supernatant was used directly for PCR amplification using primers specific for the D1 /D2 and ITS regions. Amplification using primers of the D1/D2 region was achieved using 35 PCR cycles at 94° C. for 1 min, 52° C. for 1 min and 72° C. for 1 min (Naumova et al. 2005), while the conditions for ITS region. PCR were 35 PCR cycles at 94° C. for 1 min, 55° C. for 1 min and 72° C. for 1 min (Baldwin 1992). Amplified DNA was checked on 1% w/v agarose gel and purified using the PEG-NaCl method. The purified PCR product was rechecked on 1% w/v agarose version 3.1. Sequences acquired were quality checked manually. CromasPro version 1.34 was used for contig formation and quality trimming.

### Phylogenetic Analysis

The contig obtained was deposited in the NCBI Genbank database under the accession no HQ222812. Sequences homologous to BY2 were obtained using NCBI BLAST. Sequences with high query coverage and homology were selected for phylogenetic analysis. Multiple sequence alignment was done using ClustalX (Larkin et al. 2007), the aligned sequences were trimmed using DAMBE, and the phylogenetic tree was constructed using MEGA version 4 (Tamura et al. 2007).

### Example 2

### Culture Condition/Maintenance of Microbe/Inoculum Development

10 ml of MXYP broth (0.3 g % malt extract, 0.3 g % yeast extract, 0.6 g % mycological peptone, 2 g % D-xylose) was inoculated with Pichia strain BY2 (Pichia caribbica). After 24 h incubation at 28° C., 180 rpm it was added to 90 ml MXYP broth and incubation was continued further for 48 h

### Fermentation of D-Xylose

The cells were harvested from MXYP broth after 48 hours by centrifugation at 5000 rpm, 20 mins, 10° C. These cells (wet weight approximately 4.0 g) were then re-dispersed in 100 ml of 10% xylose solution (4% cell loading). The incubation was continued further till 72 h.

### Product Harvesting

The cells were separated by centrifugation at 5000 rpm, 20 mins, 10° C. The bright yellow coloured supernatant was subjected to concentration by rotary vacuum evaporation. Temperature of water bath was maintained at 85° C. The cells obtained after fermentation can be reused for up to 3 times without substantial loss of efficiency.

Concentration of product: Bright yellow viscous fluid was collected after rotary vacuum evaporation. Further it was concentrated by centrivac (LABCONCO centrivap concentrator) system. The final yield achieved was 8.5 g/10 gm of xylose in 100 ml with 99.9% purity.

### Decolorization

Decolorization was done using absolute ethanol. 5 ml of absolute ethanol was added to 2 g of semi-crystalline yellow coloured material. Over the period of 24 hours, the material was washed thrice with absolute ethanol and after overnight contact; powder was again washed with the solvent. The intense yellow colour got eluted from the crystals into the solvent (ethanol) layer. The solvent was discarded and residual solvent was allowed to evaporate at room temperature (25° C.) to get complete dry white crystalline powder.

### Example 3

### Characterization of Xylitol

Complete characterization of the synthesized product was done with the help of XRD, H¹ NMR, FT-IR, HPLC and SEM. Purity of the compound was specifically determined by HPLC and H¹ NMR.

1) H¹ NMR: Two milligrams of samples were dissolved in 0.5 mL of DMSO. 1H NMR (200 MHz) spectra was recorded by Bruker AC200 at 25° C. Chemical shift was expressed in ppm. Deuterated methanol was used as the solvent. Tetramethylsilane was used as an internal standard.

FIGS. 1 (a) and 1 (b) show H¹ NMR spectra of Sigma Xylitol and Pichia synthesized Xylitol respectively.

2) FTIR analysis: After separating the white crystalline compound from the spent broth it was subjected to preliminary identification by FTIR. The crystals were crushed with KBr, pelleted and the Fourier transform infra-red (FTIR) spectra were recorded on a Perkin-Elmer Spectrum One in the frequency range of 4000 to 500 cm−1. Additionally FTIR spectra of pure xylitol from sigma and pure xylose were also recorded.

The Fourier transform-IR spectra of Pichia xylitol and pure Sigma xylitol were recorded and are presented in FIG. 2. The IR spectra represents a broad stretching around 3200-3400 cm⁻¹ which is characteristic of hydroxyl group present in this sugar alcohol and a weak C—H stretching band at around 2932 cm⁻¹ for both experimental and sigma xylitol. The absorption band from 1300 cm⁻¹ to 800 cm⁻¹, called “finger print” region, and is related to conformation and surface structure of molecule. These bands have always been very hard to explain however in the spectra it can be seen that, both the compounds, Pichia and Sigma xylitol show essentially similar peaks in the fingerprint region also. There was a strong characteristic peak around 1410 cm⁻¹ and 2931 cm⁻¹ which is typical of methylene groups present in the molecule. Thus, on comparison of both the spectra the compound was found to be xylitol.

3) X-Ray Diffraction analysis: To further confirm the identity of crystal X-ray diffraction (XRD, Philips X'Pert PRO) was done. For XRD analysis the crystalline sample was crushed to a very fine powder in a mortar and pestle. It was then filled in a 1 cm by 1 cm in size and 1 mm deep square etched on a glass slide. The glass slide was carefully placed in an empty petri dish, taken to the X-ray diffractometer and its spectra was recorded. The diffraction pattern obtained was then matched against the standard JCPDS-PDF database to confirm that the crystals were that of xylitol.

FIG. 3 shows the graph representating of the XRD data On comparing the experimental spectra against the standard database, it was evident that the white crystalline compound is indeed xylitol (JCPDS no. 34-1802).

4) Scanning electron microscopy (SEM): FEI Quanta 200: 3D was used to visualize the crystal morphology. The powder sample was loaded on to carbon wafers and visualized.

FIG. 4 shows the crystal morphology of Pichia synthesized xylitol as seen under the scanning electron microscope. The crystal morphology is indicative of the purity of the compound and is similar to that reported previously (Hongxun Hao et. al). The shape of the crystals appears rounded and there is agglomeration in some areas which can be neglected.

5) HPLC analysis: HPLC was also performed to know about the purity of produced xylitol according to the method mentioned by GyanPraksah et al. (2011). The experimental sample and sigma xylitol as a reference were dissolved in milliQ water (10 mg/ml) were analyzed by high performance liquid chromatography (HPLC) system of Chromeline-Hitachi. The column used was Waters sugar pak 6.5×300 mm. The mobile phase used was milli-Q water with 100 μM EDTA and 200 μM CaCl2. The flow rate was maintained at 0.4 ml per minute and column temperature was maintained at 70° C. The sugar and sugar alcohol were detected with the help of Chromline L-2490 refractive index detector.

HPLC analysis revealed that Pichia synthesized xylitol showed similar retention time as that of standard Sigma xylitol under identical conditions. Also after analyzing the spent culture supernatent for residual xylose content it was observed that almost 90% of the xylose in the medium had been fermentatively converted into xylitol which is in accordance with yield of xylitol.

### Example 4

### Violacein Inhibition Assay (Qualitative)

This assay has been designed in such a way that a zone inhibition of Violacein synthesis can be seen around the agar well containing the probable QS Inhibitor. Chromobacterium violaceum mutant CV026 is used as the test organism which has the ability to respond to a variety of signal molecules. The QS signal molecule (12.5 μM) is added to 10 ml of Luria bertani soft agar containing 100 μl of overnight culture of CV026. The soft agar is then overlayed onto basal LA plate, and a 4 mm diameter well is dug in the centre of the plate using a sterile cork borer after the overlay is set. To the agar well 75 μl of appropriate concentration of the compound under investigation was added, and the plates were incubated at 30° C. for 24 hours. The diameter of zone of Violacein inhibition was then measured. Pure xylitol from sigma was used for comparative analysis.

### Violacein Inhibition Assay (Quantitative)

This assay is based on a similar principle to the one described above, that is the presence of any quorum sensing inhibitor will quantitatively decrease the production of the purple pigment violacein which can then be estimated colorimetrically. This is a tube assay in which 100 μl of overnight culture of CV026 was inoculated in 10 ml LB broth containing the QS signal molecule (namely C6-HSL, C8-HSL or 3-oxo-C6-HSL) at appropriate concentrations. The test compound, Pichia sp. synthesized xylitol in the present invention, was then added at increasing concentrations in a series of test tubes. The violacein produced was extracted from the culture broth by dissolution of the pigment in DMSO and separating it from the cell mass by centrifugation. The amount of xylitol required to inhibit or substantially decrease the purple pigment production could be quantitatively estimated by measuring the optical density at 570 nm. Appropriate positive and negative controls were used. All experiments were done in triplicates for checking reproducibility.

### Result

FIG. 5 shows the zone of inhibition of Violacein synthesis around the agar well, containing Pichia xylitol. In both qualitative and quantitative assay it was observed that xylitol could cause receptor antagonism and hence inhibition of quorum sensing associated phenotype in CV026. From the graph in FIG. 6 it can be seen that Pichia xylitol could quantitatively inhibit Violacein production in CV026. It was seen that increasing the concentration of xylitol was not inhibitory to the organism but it severely impaired quorum sensing signal reception.

Thus, the fermentation process disclosed for the preparation and separation of crystalline xylitol from its fermentation broth using improved downstream processing to produce 99% pure crystalline form reduces the cost and improves recovery of the final product.

### ADVANTAGES OF THE INVENTION

Advantages of instant invention are as following:
1. 99% pure crystalline xylitol is obtained with almost quantitative yield.
2. Simple, mild and cost effective downstream processing with improved recovery of final product
3. Xylitol yield obtained is 85 g/100 gm, which is close to the maximum reported till now using yeasts as microbial factories.
4. Pichia caribbica BY2 synthesized xylitol can be used as a quorum sensing antagonist in gram negative organisms.
5. The novel yeast species, Pichia caribbica BY2, is capable of production of xylitol from xylose and other pentose sugar containing raw materials such as hemicelluloses, however depending upon the composition of raw material, additional downstream processing steps may be required to achieve crystalline product.

1. A room temperature fermentation process for the synthesis of crystalline xylitol from D-xylose using yeast strain Pichia Caribbica BY2 having MTCC No. 5703 comprising the steps of:
a. inoculating 10⁸ cells per ml of MXYP (malt extract xylose yeast extract and peptone medium) with yeast strain Pichia caribbica BY2 and incubating at temperature in the range of 25 to 28° C. in a rotary shaker at 170 to 180 rpm for period in the range of 20 to 24 hours to obtain inoculated MXYP broth;
b. adding the inoculated MXYP broth as obtained in step (a) into 90 ml MXYP broth and further incubating for period in the range of 40 to 48 hours to get sufficient cell biomass;
c. harvesting cells from MYXP broth by centrifugation at 4500 to 5000 rpm for period in the range of 15 to 20 minutes at temperature in the range of 4 to 10° C.;
d. re-dispersing the cells from step, (c) in the ratio ranging between 3 to 4 g % of 9.5 to 10% D-Xylose solution and incubating further for period in the range of 70 to 72 hr;
e. separating cells by centrifugation at 4500 to 5000 rpm for period in the range of 15 to 20 mins at temperature in the range of 4° C. to 10° C.;
f. concentrating the yellow coloured supernatant obtained in step (e) by rotary vacuum evaporation in a 85° C. water bath to obtain the bright yellow viscous fluid;
g. further concentrating the bright yellow viscous fluid obtained as obtained in step (f) by centrivac concentrator system;
h. crystallizing the concentrated bright yellow viscous fluid as obtained in step (g) by refrigerating at temperature in the range of (−)15° C. to (−)20° C. for period in the range of 40 to 48 hours, to obtain semi-crystalline yellow material;
i. decolourizing the semi-crystalline yellow coloured material as obtained in step (h) using absolute ethanol to obtain crystalline xylitol with 0.8 to 0.9 gm/gm yield and 98 to 99% purity.

2. The process as claimed in claim 1, wherein the MXYP broth used for inoculating Pichia caribbica BY2 contains Malt extract 0.3 g % to 0.33 g %, Yeast extract—0.3 g % to 0.33 g %, Mycological peptone,—0.6 g % to 0.65 g % and D-Xylose—2 g % to 2.5 g %.

3. The process as claimed in claim 1, wherein said crystalline Xylitol acts as a quorum sensing antagonist that prevents the bio film formation by gram negative bacteria using anti-AHL (Anti-acyl homoserine lactone) activity of xylitol.

4. The process as claimed in claim 3, wherein the gram negative system is preferably Chromobacterium violaceum CV026.

5. The process as claimed in claim 1, wherein said xylitol inhibits Quorum sensing signal (acyl homoserine lactone) reception by three distinct signal molecules selected from C6-HSL, C8-HSL and 3-oxo-C6-HSL in gram negative bacteria.

6. The process as claimed in claim 5, wherein said xylitol acts as a quorum sensing antagonist in Burkholderia cenocepacia having two distinct signal molecules, C6-HSL and C8-HSL or in Erwinia carotovora having one signal molecule, (3-oxo-C6-HSL).

7. The process as claimed in claim 1, wherein room temperature is in the range of 25 to 28° C.
